(19) United States
(12) Patent Application Publication
(10) Pub. No.: US 2011/0077499 A1
(43) Pub. Date: Mar. 31, 2011

(54) Methods and apparatus for improved diagnoses and oncological treatment and treatment planning

(76) Inventor: John Joseph Pagani, IV, Houston, TX (US)

(21) Appl. No.: 12/586,942

(22) Filed: Sep. 30, 2009

(51) Int. Cl. A61B 5/05; G06Q 50/00; G06K 9/00

(52) U.S. Cl. 600/407; 705/2; 382/128

(57) ABSTRACT

Methods and apparatus are disclosed for improving the diagnoses of abnormal conditions, including tumors, damaged tissue and improperly functioning body systems, as well as methods and apparatus for providing virtual pathology/surgery and improving the planning for and execution of oncology treatment. A typical body scan produces only two-dimensional images, in varying shades of gray, and may typically provide either little or no information about structures intermediate the standard image slices: the methods and apparatus disclosed may be used to autonomously prepare from such a series of two-dimensional images a three-dimensional image of structures or systems of interest, which may then be rotated, oriented, and sized as desired; the structures or systems of interest—whether those which should receive treatment or additional treatment or those which should be spared—may be highlighted in color or different colors. A physician or other treatment specialist may then instantly see an entire structure of interest, even as to the volumes intermediate the standard two-dimensional images. Selecting a structure or system of interest may produce not only identifying labels but robust textual descriptions of structures and functions, audio presentations of important relevant information, and even video and audio presentations to aid in the diagnosis and treatment of movement disorders.

### BACKGROUND OF THE INVENTION

This invention relates generally to methods and apparatus for improving the characterization and/or diagnoses of abnormal conditions such as tumors, damaged tissues or improperly functioning body systems, to methods and apparatus for providing virtual pathology/surgery, and to improving the planning for and execution of oncology treatment and surgery. It is particularly useful for displaying proper reference anatomy for all forms of anatomical variations, whether the cause or source of such variations be disease-related, treatment-related, surgical-related, normal, genetic or physiological variations.

The handicaps under which treating oncologists work today are simply staggering to those not intimately acquainted with the field. For many structures of the body, there is little if any useful visual information available to the treating oncologist in the critically-needed sectional format; in many instances, the information is available only in written descriptive texts or in views convenient for surgeons but not for imaging oncologists. In many other instances, the black-and-white pictorial or schematic representations are so small and non-detailed that they provide scarcely more useful information than a textual description. Even when decent reference images are available, they are virtually always presented in two dimensions (2D) only, are very few in number, and are limited to a “standard” reference which all too often is of little real help to the treating or planning physician.

Further, until the recent advent of diffusion tensor imaging (DTI) technology, medical science had no means of imaging many of the functional systems of the brain, for example, the auditory system, the olfactory tract, speech, hearing and visual networks, motor tracts and the like. This is to say, to X-ray imaging, CT scans, MRI scans, PET scans and the like, such “circuits” are simply invisible, and few physicians have access to DTI technology, which remains quite expensive. Consequently, planning physicians today have no adequate learning tool which displays the locations of brain circuits of interest on each of the consecutive sectional images in which they should appear; rather, all that the physician can rely upon are a few imaging examples, autopsied (and desiccated, non-functioning) brains, and inadequate schematic drawings. When such a functional circuit is invisible, present standard procedure requires the physician to simply guess at the exact locations by plotting educated guesses from known internal reference points: for example, it is known that motor tracts (usually) travel inferiorly from the precentral gyms where they originate to the lateral ventricle, so the physician may guess at their locations by drawing a smooth curve, or plotting a locus of sequential points, from one such reference point to another. The lack of precise information as to the actual path normally results in the planning physician's demarcation of a larger area to be protected from radiation than would be necessary were more accurate information available, or even the demarcation of a wrong area to receive radiation. This may, of course, result in inadequate irradiation of tumors or pre-cancerous areas, which in turn may result in the death of the patient.

The range of physiological variations among ‘normal’ or at least healthy humans is enormous: the origins and paths of many vessels differ greatly, yet no useful references are available which a treating physician can readily call upon which are capable of displaying such great variations in the standard or reference images. Both the diagnosing and treating physicians need to know the paths and origins of vessels for any particular patient. As might be expected, the situation regarding standard reference variations is even worse for disease-related, treatment-related, and surgical-related variations.

Few if any references are known to be capable of presenting useful visual images on demand which depict body systems as normally affected by congenital variations, trauma, disease, treatment or surgery. One common cancer condition is a collapsed lung among a segment of the population which cannot withstand conventional treatment to re-inflate the affected lung. With no known radiotherapy references which accurately depict how a sectional image of a collapsed lung looks, standard treatment for such a patient is highly problematic, at best. Recently, for example, a prominent oncologist demonstrating his treatment process for such a patient at a nationally-attended conference mistook the mass of a collapsed lung for the tumor he intended to treat: consequently, the patient's healthy but collapsed lobe received a substantial dose of radiation which was not needed, and if the tumor which was the intended target received any radiation at all, it was purely coincidental. The consequences of such mistakes, which arise from a lack of adequate information, are readily apparent.

It has long been known that numerous diseases may grossly affect the imaging appearance of various organs and other body parts, yet treating physicians today have no organized, unabridged guide to the appearances of such affected organs or other parts, and in some instances may not be familiar with the normal appearance of a particular type of tumor. A pancreatic tumor, for example, does not look like most other tumors, and often will initially manifest itself by blocking and thereby enlarging the pancreatic duct, common bile duct and cystic duct as well. Not only will a pancreatic tumor affect the appearance of the pancreas, but it may significantly increase the size of the gall bladder ducts as well, yet there is no reference capable of presenting all this long-needed information conveniently and on demand.

Similarly, there is no known reference which can produce proper post-surgical references upon demand, or which can combine images expected from changes caused by disease with those expected from surgery. The stomach is typically illustrative: cancer of the stomach can increase the thickness of the wall of the stomach from 3 mm to 7 mm, and at least five different types of surgery to re-connect the stomach remnant to the intestine can be performed with significantly differing effects upon post-operative imaging and radiation planning. An accurate mental picture of the complicated or “spaghetti-like” loopings of the reconstructed bowels is an absolute imperative for accurate radiotherapy planning, yet accurate interpretation of the surgical changes presented on the standard serial sectional diagnostic images used for radiotherapy planning borders on the impossible, even when written operative notes are provided. Such sectional images inherently show only a small part of the complex surgical realignment, and building a mental 3D-image of multiple bowel loops extending in six possible directions is quite difficult, even when the surgery is understood; without a firm 3D understanding of the reconstruction, it is virtually impossible. Currently, the state of the art for sorting out bowel loops is quite restricted. The physician may have the patient drink barium and observe its transitory progress through a fluoroscope, or he may refer to a series of sectional 2D images among the three planes, axial, coronal and sagittal; there is no overall 3D sectional imaging available. The procedure is inherently prone to error, and even when done accurately—or particularly when it is done accurately—the procedure is extremely time-consuming. It is fair to describe the state of the information available in this particular sub-field as very complex and poorly understood by most imaging oncologists. Knowledge of these different types of surgeries is usually kept in diagrams presented from the surgeon's viewpoint and not from the imaging oncologist's viewpoint, making it so difficult as to be nearly impossible for the oncologist to properly develop his radiation treatment plans on 2D sectional images.

Post-treatment variations can range from minor but still consequential to highly significant. A case in point is prostrate treatment, wherein a treating physician typically seeks to immobilize the prostate during treatment; immobilization is normally accomplished by inserting and then inflating a balloon in the rectum. This procedure, however, will shift at least the positions if not the shapes of not only the prostate but also the prostate nerve vascular bundle, the rectum itself and the anus and anus sphincter. It is important for the treatment planning physician to know that these changes are the ‘normal’ results of the treatment and not due to some hidden and potentially dangerous cause. Also, treatment of a tumor normally results in change of the size and shape of the treated tumor, which requires the implantation of (typically metal) markers to serve as fixed reference points to permit the changes in the tumor to be tracked and to insure that subsequent treatments are directed to the proper region.

The present invention overcomes these severe handicaps by a number of different means. First, diagnostic ability is radically improved by the inclusion of various videos presenting various overt manifestations of abnormalities, which often are subtle enough to be missed in conventional clinical examinations. An audio/video library of speech abnormalities, for example, which can be viewed while simultaneously examining X-ray or other images, allows diagnosis of the specific region of the brain affected. A video library of movement disorders has application across a wide array of physiological problems. Tremors of Parkinson's disease exhibit certain frequencies, and different frequencies, for resting tremors and attention tremors; a video library of such movement disorders allows the earlier and more certain diagnosis of the condition, and earlier treatment thereof. Also, specific lesions of the brain cause different gaits; the diagnosing physician can view the video library while observing a particular patient's gait and match the particular gait, thereby learning what lesions are present, where. Additionally, videos of eye-movement disorders, when matched to a particular patient's eye-movement disorder, will show the diagnosing physician what part of the brain is affected. The art currently aids diagnosis of speech disorders typically with only written descriptions of such disorders; a speech disorder audio library allows rapid (and more certain) diagnosis of the affected region of the brain.

With scant input such as a few gray 2D images of structures of interest from CT scans, MRI scans, etc., the present invention can convert the 2D images into 3D images of structures of interest and continue such structures—whether structures to be treated or structures to be avoided—throughout the volume intermediate the few input scans, while allowing it to be rotated at will and viewed from any angle, thereby greatly enhancing understanding of the structure of interest.

### SUMMARY OF THE INVENTION

A notable feature of the invention is a computer-searchable database which has converted a vast amount of highly disorganized, difficult-to-access descriptive textual information and difficult-to-understand, poorly-presented gray flat images, for each field and sub-field of the human body, into immediately accessible, dynamic 2D and 3D images. Indeed, it would not be an exaggeration to describe the state of accessible medical information today as chaotic, which undoubtedly should be expected in view of the random and happenstance manner in which such information came to be gained over the preceding centuries. In all too many instances there is simply no linkage between important fields which can significantly affect each other, e.g., bronchoscopy literature and images used for lung irradiation; the database has not only compiled the information in each field but has correlated it with each other, and the associated computer program presents only what is needed to plan and treat any stage of a given cell type of tumor. The database further comprises both ‘normal’ images for a wide range of ‘normal’ humanity and altered images resulting from trauma, disease, and surgical or other forms of treatment; this allows a diagnosing and/or treating physician to rapidly and accurately understand whether an apparently non-standard image being viewed is indeed within the bounds of normalcy for a patient who has experienced what that particular patient has experienced or whether it is indicative of a suspicious region which merits deeper examination.

In addition to creating a 3D image from a series of standard 2D ‘slice’ images, the 3D-created image is dynamic, meaning that it can be sized as appropriate and rotated at will, and structures of interest, such as any particular brain circuit, can be displayed from whatever viewing position is most conducive for comprehensive understanding and their locations simultaneously identified on a 2D map which can be overlaid accurately onto a patient's scan. The program and database cooperate automatically to display both the region of the brain containing the volume to be treated and the various brain circuits to be protected. Some of the functions which are not currently fully protected under the present state of the art are vision, memory and limb movement; some functions which enjoy no protection whatsoever include motivation, organization, social behavior, eye movement, hearing, smell, equilibrium, facial sensation and movement of the facial muscles. Each portion of a pathway can be highlighted on a 2D image and instantly labelled with informative text and color-coded through all the 2D sections, and its entire path shown in a companion 3D image. Alternatively, all brain tracts can be shown simultaneously and in both 2D and 3D and the user may select only the ones of interest. By overlaying the tumor requiring treatment into the 2D image stack, the invention facilitates custom mapping of the regions to be avoided or treated: if a tumor is damaging a circuit which is present on both sides of the brain, the redundant circuit on the opposite side should be protected in order to prevent loss of function.

Subsequent to a gastrectomy (removal of the stomach) or partial gastrectomy and re-attachment of the intestine, it is commonly desired to irradiate both the lower portion of the esophagus and the upper portion of the re-attached intestine in order to kill any small cancerous cells which may have spread beyond the stomach. The database contains a library of anatomically accurate 3D displays of each of the numerous types of surgeries which may have been performed; the planner may, by referring to the surgical notes, call up the proper images for that particular type of surgery. The invention then provides the critically necessary linkage between the selected surgical template and the companion annotated 2D sectional images. This is to say, the database and program do not display the effects of the surgery on just one or a few selected sectional images, but display the effects on a considerable number of consecutive and labelled 2D sectional images and a companion dynamic 3D image, thereby rapidly and accurately informing the user of the geometry of the reconstructed gut resulting from the particular surgery which that particular patient has undergone. Such instant informing is further aided by the ability to highlight each piece of the reconstructed gut on the 2D image in color, along with labeling instantly with informative text, color coding throughout all 2D sectional images, and showing its entire path in a companion dynamic 3D image. Without such a long-needed tool, the planning physician can rely only upon inadequate surgical and radiological sources for assistance in understanding these difficult-to-comprehend surgeries: the surgical literature is usually presented as en fosse drawings of the abdomen from the operating perspective, while the radiology literature typically captures only a fragmented path of orally administered contrast with serial overhead, usually with somewhat rotated X-ray images of the upper abdomen; in both instances, information presented in such formats is of but little use in interpreting sectional images of the bowel reattachments.

The invention improves planning decisions for non-small-cell lung cancer radiotherapy treatments by converting information from bronchoscopy nomenclature and information from the literature on tumor biology into dynamic 2D and 3D templates. Currently, this information exists in separate domains which are not linked in any meaningful way with the images used for planning lung irradiation. The present invention not only correlates this data but breaks it down into units which contain only what is needed to plan any stage of a given cell type of tumor. For example, when the tumor site, “lung,” is selected and the stage of the tumor designated, information of four different types is color coded and embedded in the normal 2D imaging studies and their companion, moveable 3D display. First, radiosensitive normal areas (such as nerves of the armpit which control the upper limb, the esophagus, et al.) are highlighted for protection from irradiation. Next, tumor site-specific nodes likely to contain tumor cells are displayed for consideration as X-ray (or other forms of radiation) target regions. Then, the imaging appearance of the site-specific bronchial tree is provided to merge the baroscopic location of the tumor into the imaging set.

The invention is of particular utility for conducting so-called ‘virtual’ surgery and ‘virtual’ pathology. Virtual surgery is real surgery, only conducted in a manner differing vastly from the days of Avicenna and Menomides: typically, a very small incision is made, and a lighted fiber-optic cable with lens inserted, along with whatever surgical tool(s) may be desired. It is, of course, critically important that the surgeon performing the procedure be able to understand just exactly what the image presented on his screen actually represents. The capability of the invention, as outlined above, to flesh out a few gray sectional slices into full 2D and 3D maps, with highly informative labels and text as needed, and abnormalities clearly pointed out, permits all such surgeons to be adequately informed before the surgery begins and to remain adequately informed throughout the surgery. One significant benefit from such use of the invention will be to raise the standard of performance for all surgeons conducting such surgery.

Virtual pathology may be thought of as the capability of performing oncology studies to a depth previously unheard of, and “on-the-fly.” This is to say, the planner is not necessarily limited to a series of static or unchanging images, but has available images which may be changed virtually at will, as may be desired. For example, the focal point of any image may be altered as desired, and 3D images rotated as preferred for maximum clarity and understanding; a particular tumor may be added, and its size and stage changed to best match a given scan, and all the variations, from disease, previous surgery or whatever, instantaneously presented to the user. In sum, the present invention tells the diagnosing/planning/treating physician what he needs to know, when he needs to know it. Widespread adoption and use of the present invention will bring the practice of medicine into the 21st century, and save countless lives and untold millions of dollars in the process.

### BRIEF DESCRIPTION OF THE DRAWINGS

FIG. 1 is

[This ‘mechanistic’ section will be completed when drawings are finalized.]

### DESCRIPTION OF THE PREFERRED EMBODIMENTS

The database portion of the present invention was created in part by searching a vast amount of poorly-catalogued and generally poorly-illustrated (or non-illustrated) references for all references which could be found that illustrated or discussed abnormal or unusual anatomical features, whether such “deviations from the norm” were due to disease, treatment, surgery, trauma or simply genetic or disparate variations among a large and highly diverse population. From this vast amount of medical literature, literally thousands were further analyzed; when drawings or sketches were found that were deemed suitable for use as starting points, a conventional “draw” program was utilized to create as many clear images as deemed necessary to embed such images into the database. When no suitable drawings or sketches could be found, archived patient scans were used as the starting points, and the features of interest hand-drawn (with the aid of the “draw” program) to embed the appropriate images into the database. All relevant textual information relating to the anatomical features or conditions so embedded were then concisely and systematically condensed into just what a diagnostician or planning or treating oncologist would need to know, and encoded for maximum convenience for the diagnosing, planning or treating professional. This is to say, any body structure can be called into view, rapidly and on demand, with or without all structures of interest and neighboring or related structures labelled, and with the concise, relevant information—“mini-encyclopedias,” as it were—presented systematically. In addition, more detailed references are also available at the click of a button or key. Should a user not want to take his eyes off a particular structure under scrutiny, he may elect to have the concise information read to him via an audio presentation.

It should be apparent that having immediately available such relevant information, in both the concise format and in the optional, in-depth format, provides a number of significant benefits. First, having such concise, precise information available literally at the fingertips of the nation's physicians will translate directly into significantly improved standards of medical care; better medical care in the short term will produce better patient outcomes, with all the attendant benefits therefrom, and better medical care over the long term will significantly increase the productivity of the nation's workforce and significantly reduce the proportion of resources devoted to providing medical care. In addition, the importance of a systematic presentation of the information thus distilled can hardly be over-emphasized; a user will not have to learn any new formats of information presentation when moving from one structure or region of the body to another, or scan through differing formats, to glean what he needs to know, when he needs to know it, thus saving the user still more time in making his diagnoses or planning or executing his treatment. Obviously, then, even such less apparent time savings will translate into significant cost savings not only for the patients but for the nation as a whole.

The pre-existing literature was also exhaustively researched for all speech disorders which could be found; in many instances, such disorders were simply described in textual format. The time savings to professionals attempting to diagnose a given patient's speech disorders are enormous; in addition to permitting the rapid matching of a given patient's disorder, the invention clearly indicates the region (or regions) of the brain which cause the disorder, helping the professional to quickly focus on the particular causative lesion(s), or, in many cases, to find lesions which might otherwise be overlooked. The literature regarding movement disorders was similarly exhaustively researched, and the highly inconvenient or barely informative textual descriptions converted into videos which easily permit the professional to quickly and accurately diagnose the cause of such movement disorders.

Use of the program is preferably initiated by presenting a number of on-screen choices for the user: patient's gender, body area of primary interest, region of tumor and type, left or right side (or midline) and cell type. Body area choices include Head and Neck, Brain, Thorax (or Chest), Abdomen, and Pelvis. Selection of any particular body area of primary interest will then preferably present the user with a wide range of choices for region of the tumor; specification of “stomach” alone provides further choice of upwards of a hundred possible combinations for stomach condition and type of tumor. In addition, for pertinent regions, the user is asked to specify “T” and “N”—i.e., the stage of the tumor and the degree of nodal involved. [Except, in the case of ‘Brain’ selection, there can be no nodal involvement.] The program will then draw the type of tumor in the region indicated and size it appropriately for the stage indicated. Should the region specified be the brain, for example, the user will also be queried re the normally-invisible but critically-important functional tracts; the user may elect to specify one or more specific tracts, or all brain tracts.

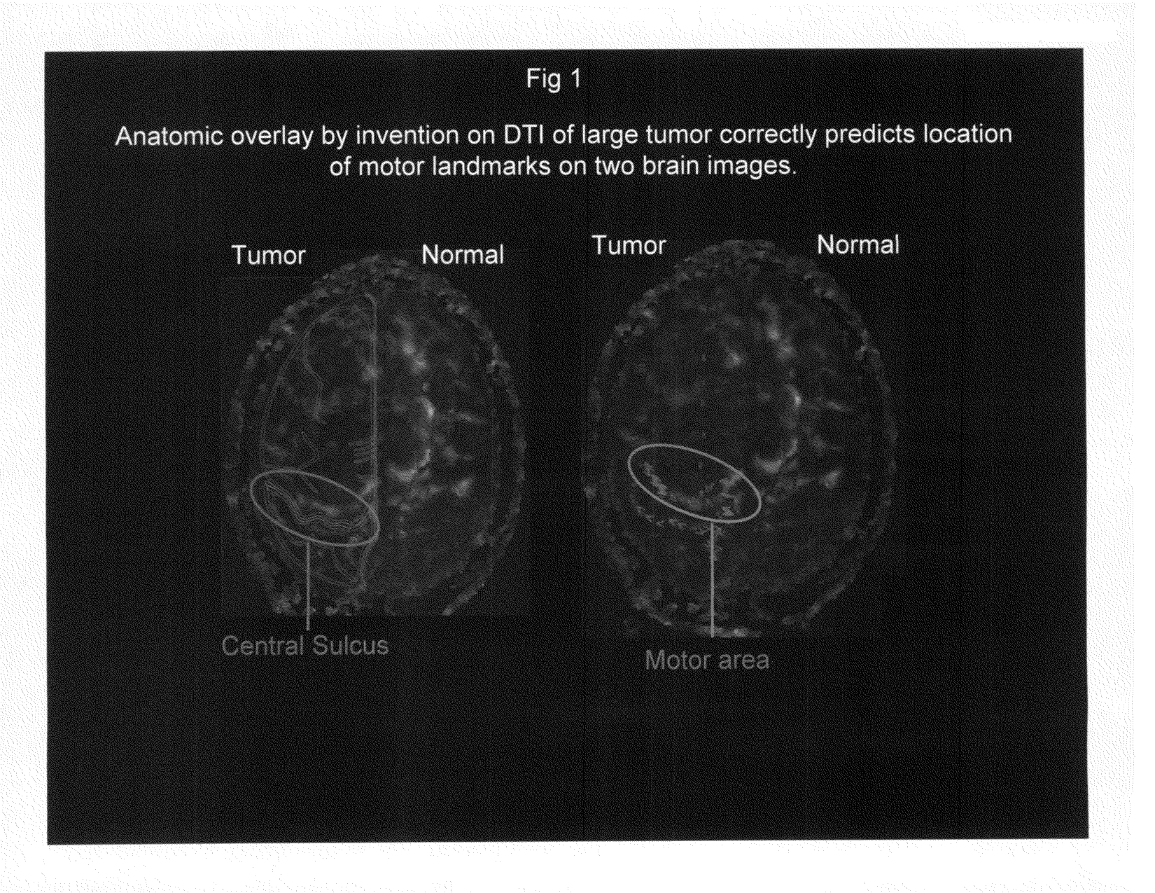

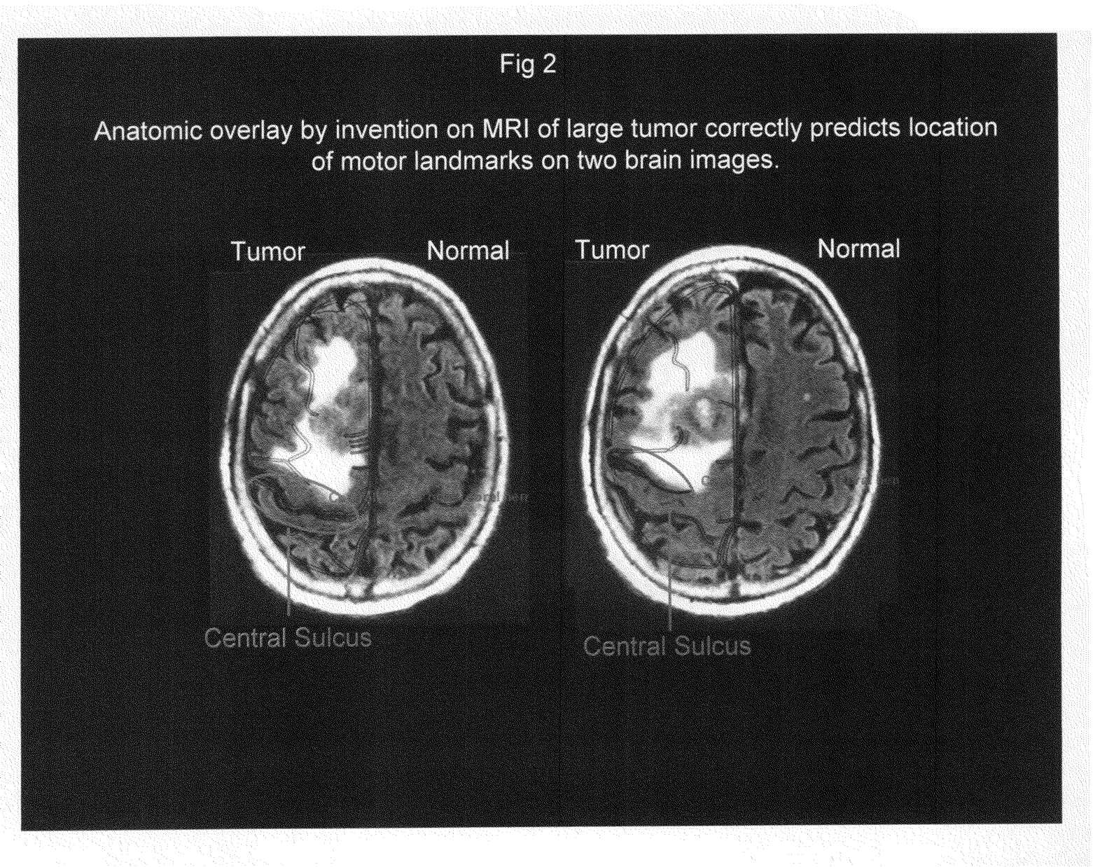

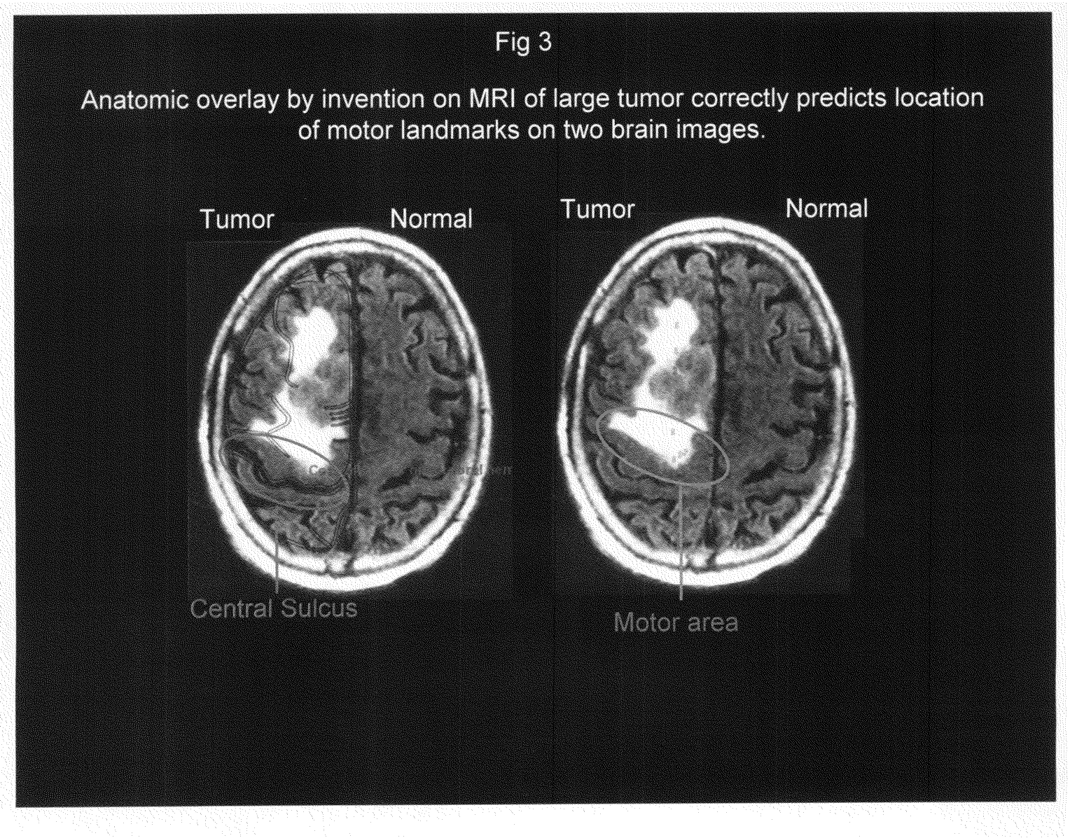

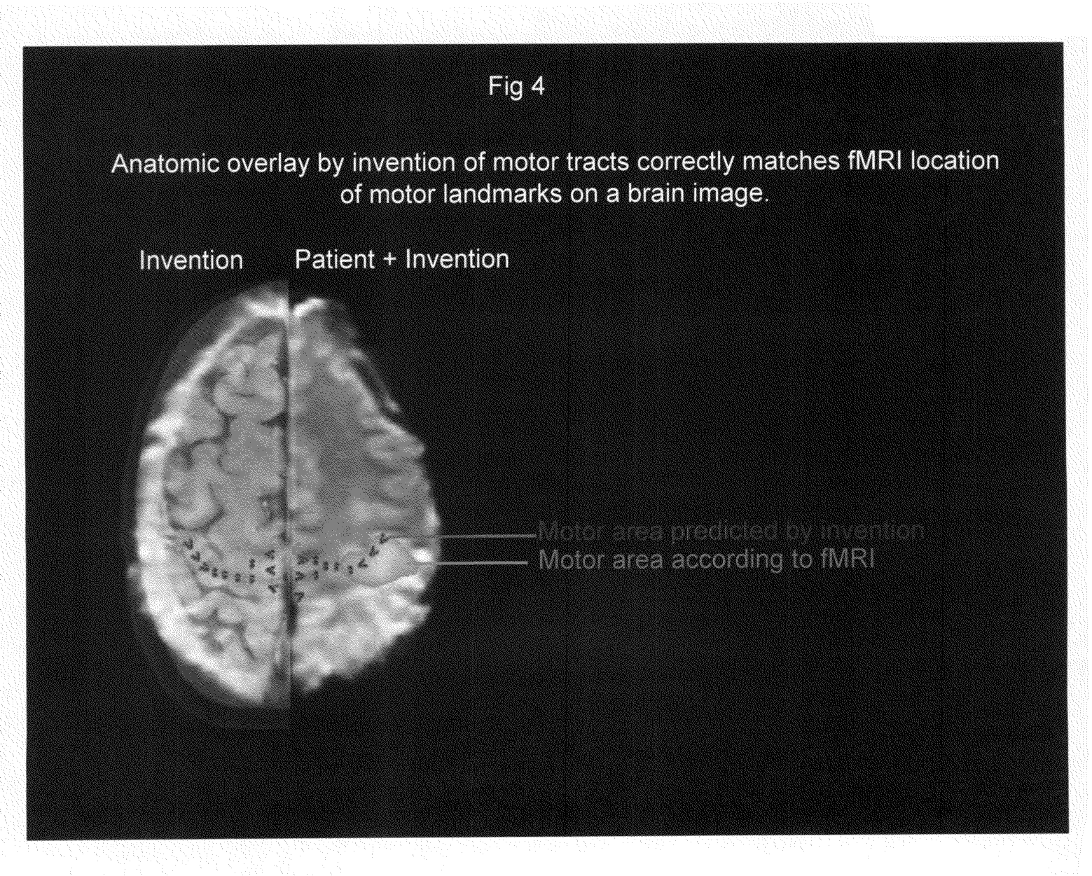

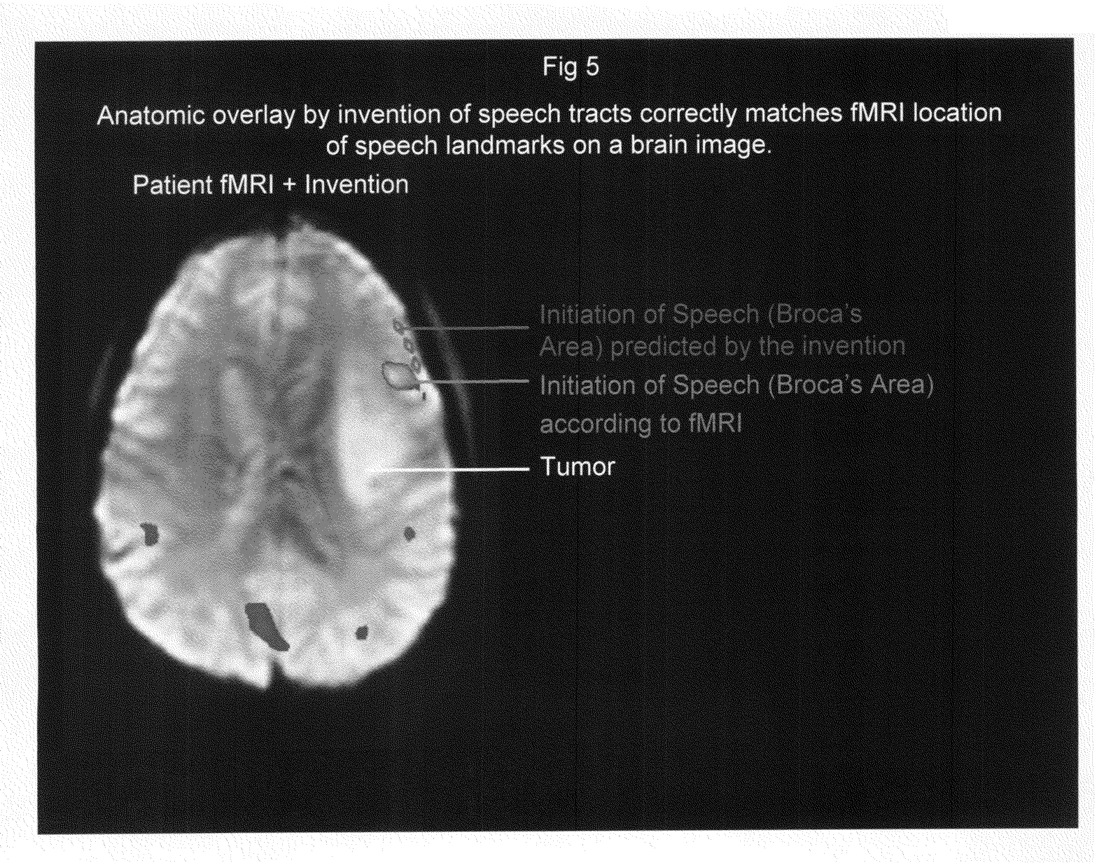

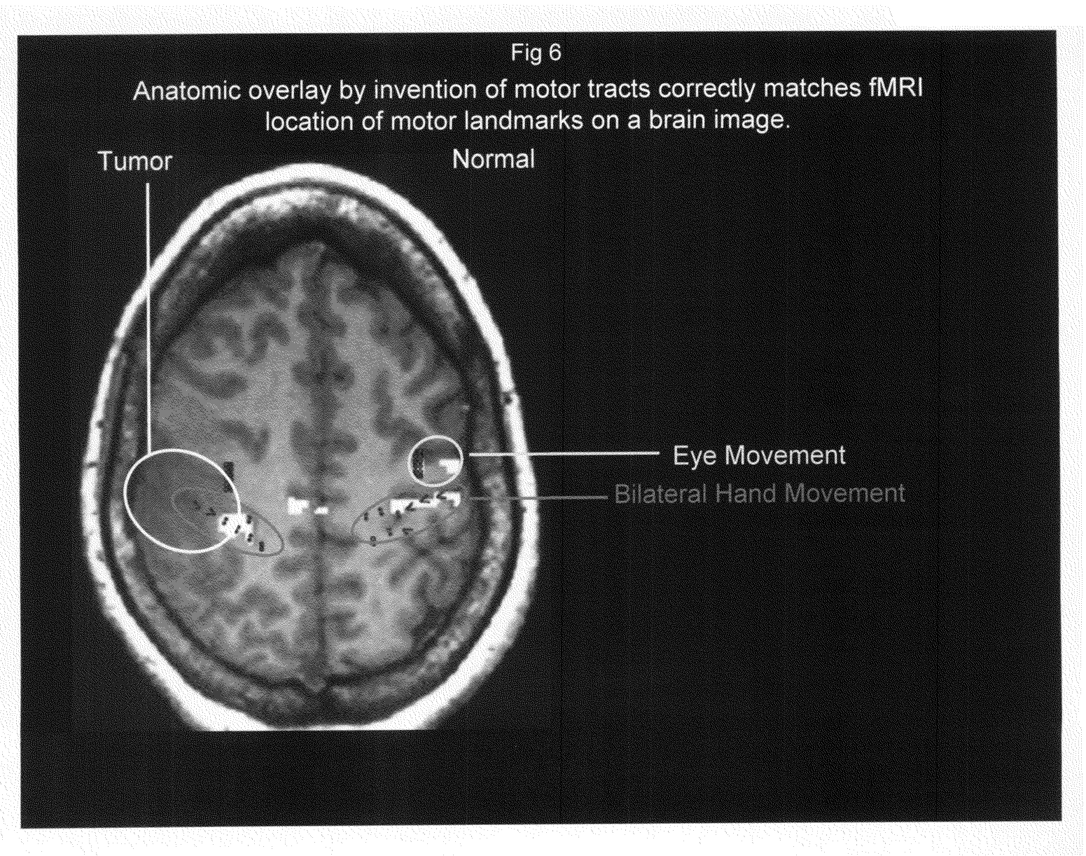

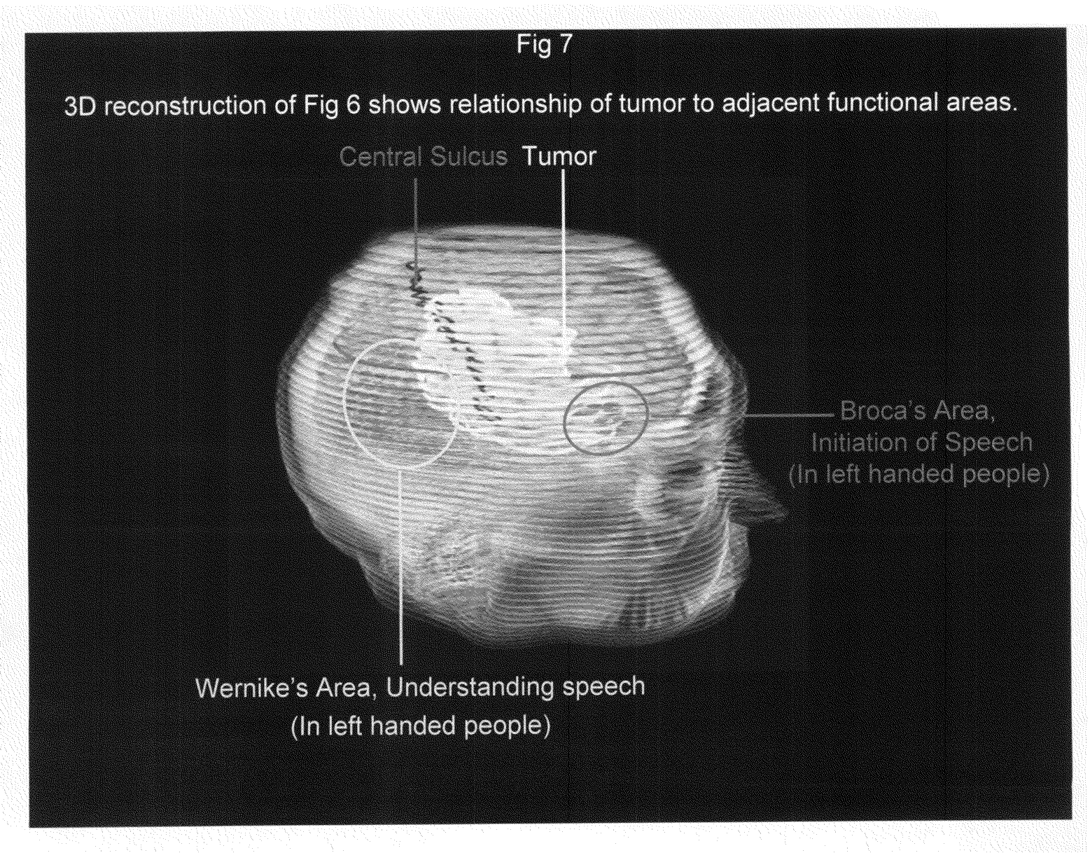

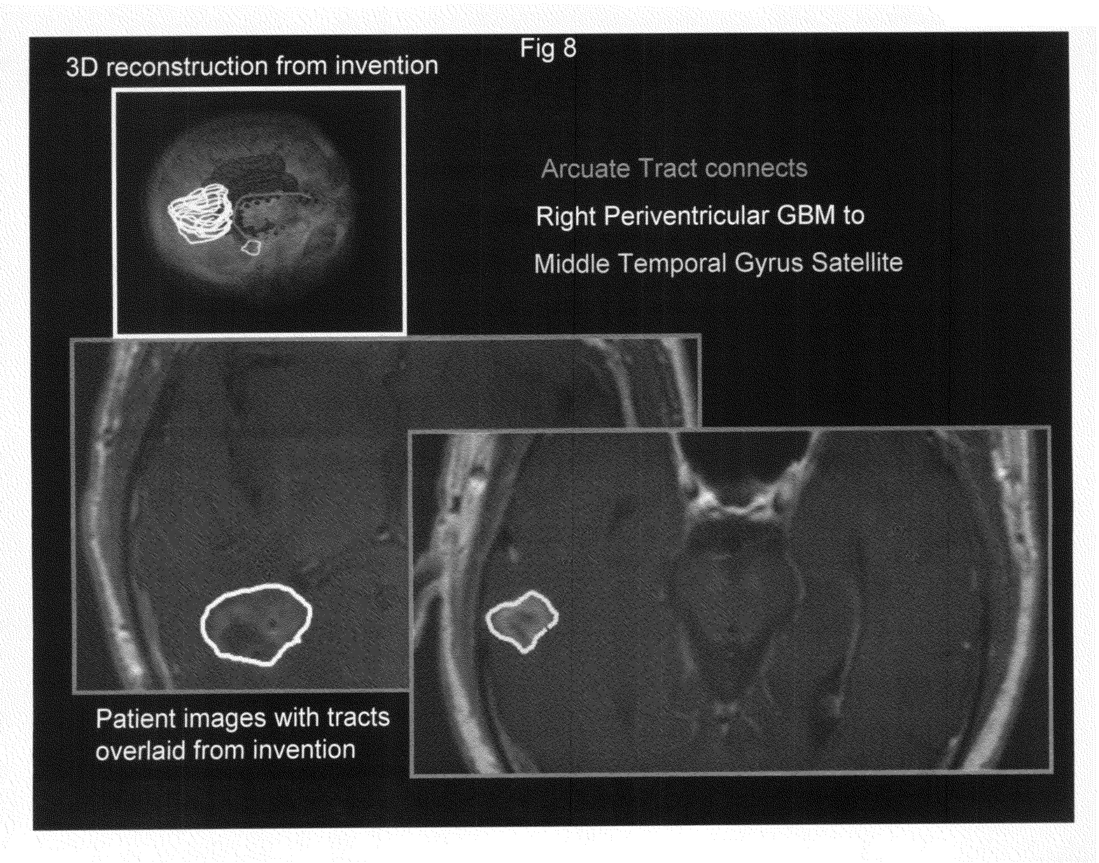

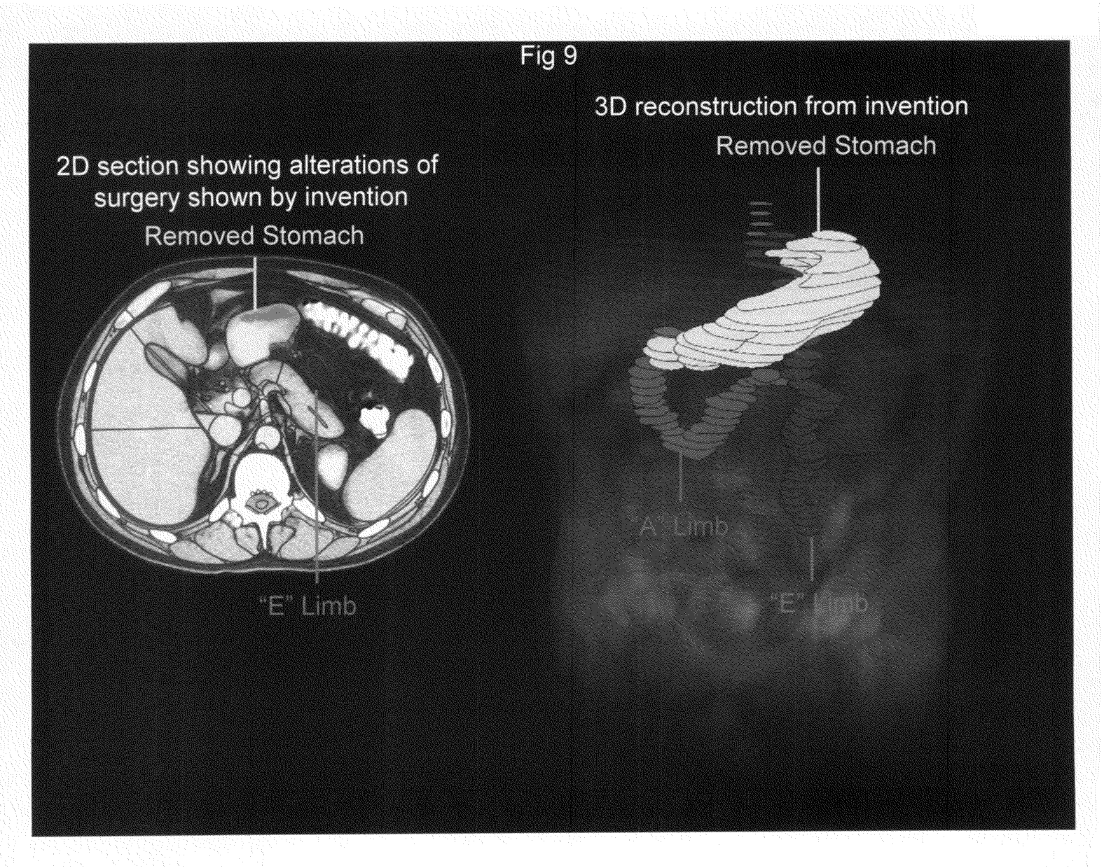

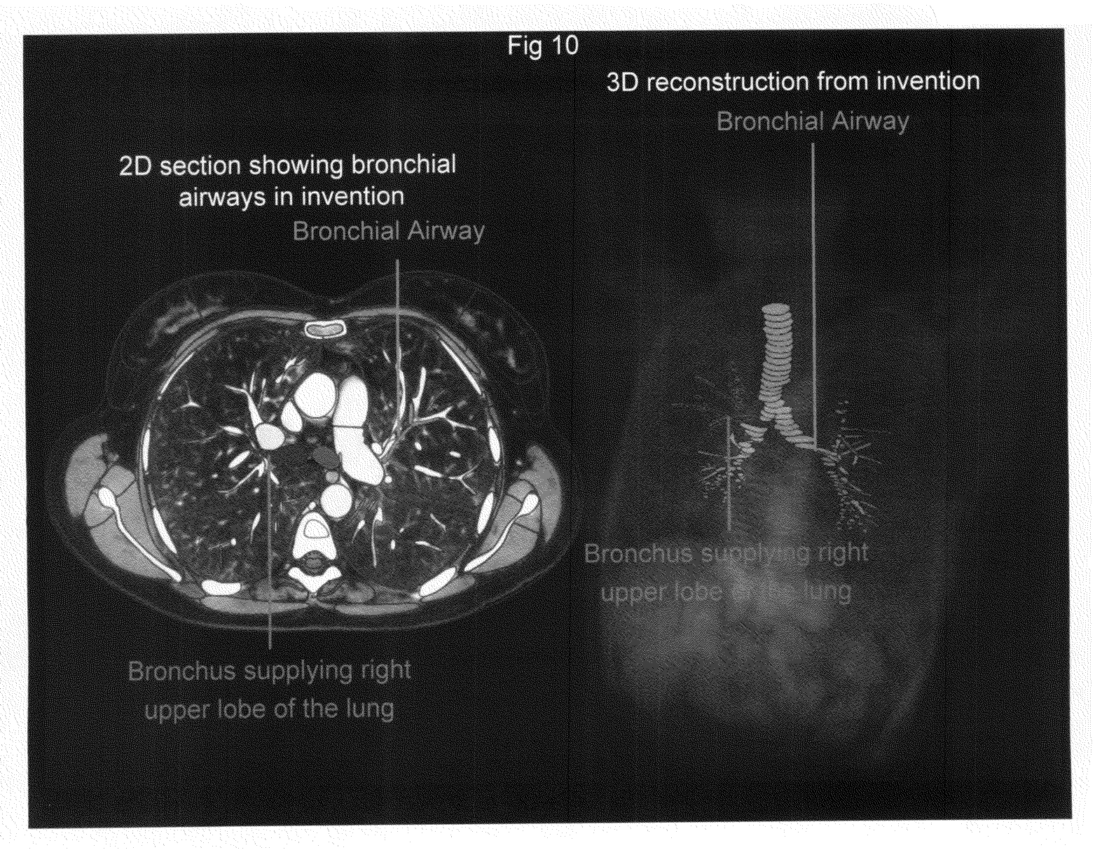

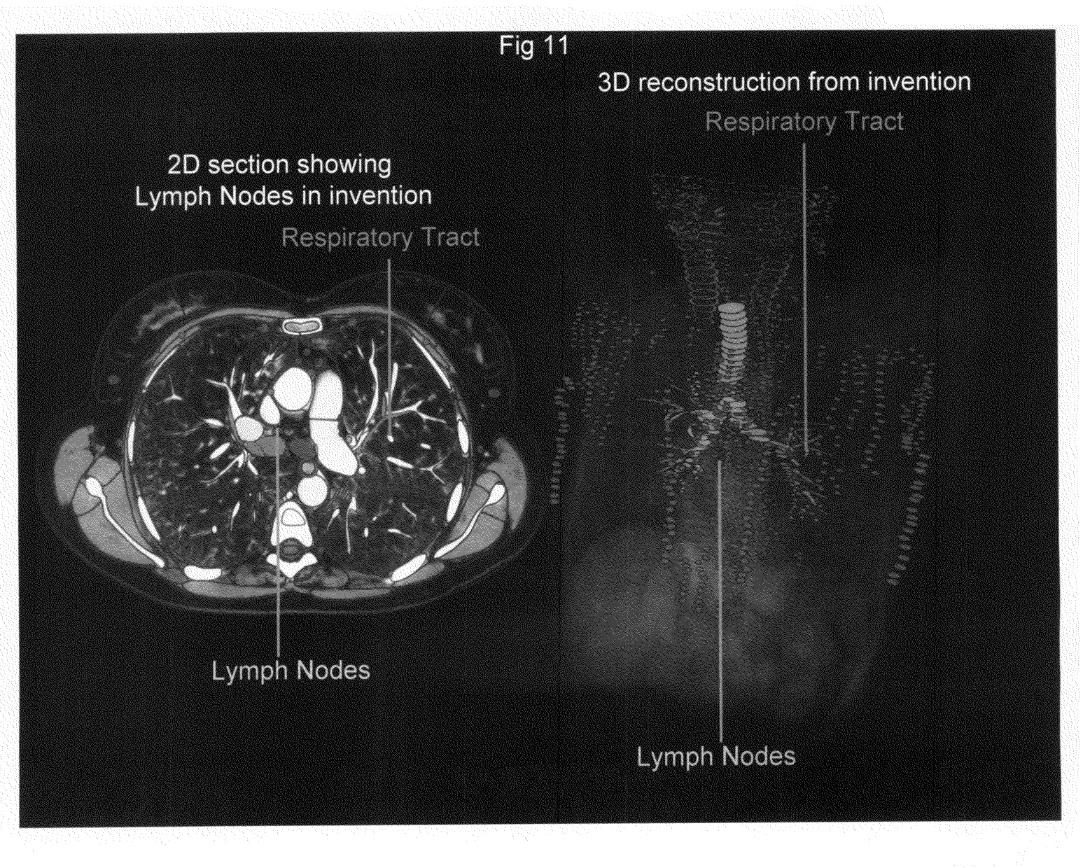

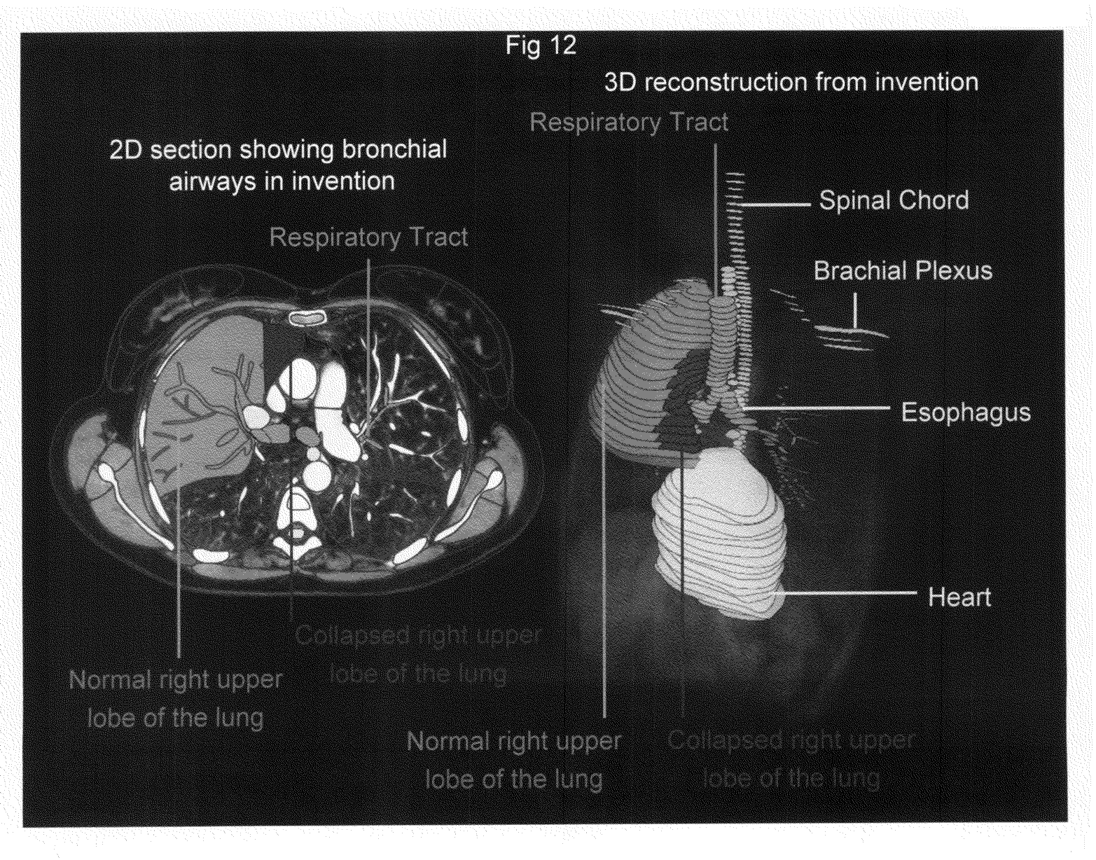

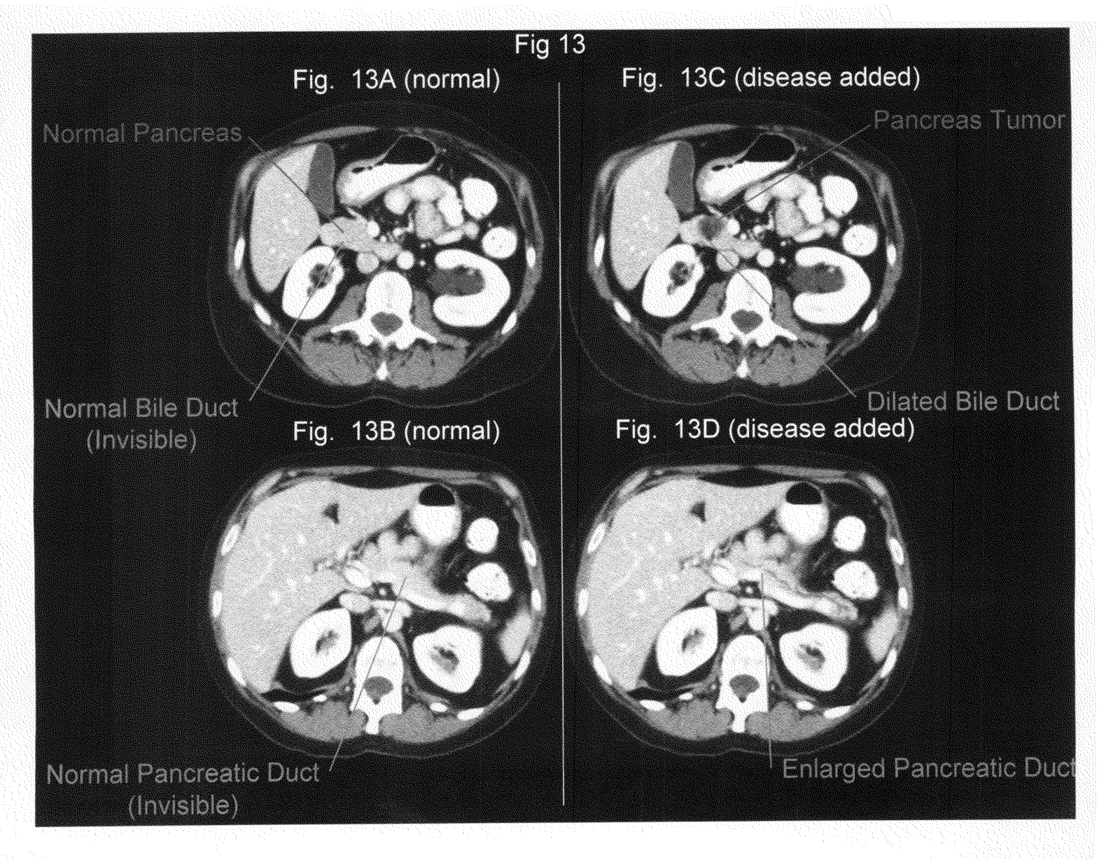

FIG. 1 is a brain image generated by the program and database of the present invention, overlaid onto a diffusion tensor image (DTI) of an actual patient with a cerebral tumor. In functional imaging, a patient is asked to perform some function, such as movement of a hand or limb; this causes water molecules in the brain to move along certain pathways, which are then highlighted in color: blue is utilized for starting points, red is used for horizontal movement, and green for vertical pathways. It is to be noted that the program has labelled critical portions of the brain which should not be irradiated, namely, the central sulcus (a motor strip of the cortex) and motor and sensory areas.

FIG. 2 depicts a succession of two sectional images or “slices” of the same patient with program overlays depicting an extremely large tumor mass (white) and showing the accuracy of the tracking of the central sulcus even near such a large mass. The short blue lines on the images show named folds of the brain (which in operable cases can conveniently serve as landmarks) even though such folds have been highly distorted by the presence of such a large mass.

FIG. 3 also depicts two sectional images of the same patient with program overlays. The tumor is again depicted in white; the motor area is encircled in orange, and a sensory area below the motor area is shown in red. It should be noted that the motor and sensory functions are not so much physical “areas” as they are tracts; i.e., pathways invisible to most imaging techniques in use today, and critical to preserve. It should also be noted that most physicians and patients today do not have access to DTI equipment, nor to functional magnetic resonance imaging (fMRI); such physicians—without the aid of the present invention—are greatly handicapped in trying to decide whether a given brain tumor is or is not operable or treatable. However, with the aid of the present invention, all physicians, even those in less populated areas who seldom see large numbers of tumors or complicated tumors, can easily and accurately conclude whether any given tumor is or is not operable or treatable by radiation.

FIG. 4 is an overlay of the present invention onto an fMRI; in response to an instruction to identify the motor function, the program has highlighted the motor regions identified by the functional MRI in yellow and located the motor tracts (or motor function pathways) with red arrow points, oriented to provide the direction of motion along such normally-invisible tracts. It is, of course, critical that these regions and tracts not be severed nor irradiated. At the option of the user, one-half of this particular image has been faded out.

FIG. 5 is a similar overlay of the present invention onto a different fMRI slice of the same patient. Broca's Area of speech initiation, according to the fMRI, has been highlighted in yellow, and that predicted by the program in red. (The green and red areas represent imaging artifacts.)

FIG. 6 is a similar overlay onto a functional MRI (of a different patient) that failed to show the cortex hand movement tract but which was identified by the program/database (purple, in orange ovals). Thus, without the present invention, such a patient would be at risk of losing hand movement from treatment, even though the patient received a functional MRI scan.

FIG. 7, for the same patient, is a similar overlay but onto a large number of horizontal slices or images; the invention traced the image of the tumor in 3D from the information in the patient's 2D scans, embedded it into the database, and created the 3D image of FIG. 7, which can be rotated and oriented at will. The program may then be queried at length about the tumor. In this instance, it may be readily seen that the glioblastoma mass (in white) has grown around the central sulcus (a motor strip of the cortex, in purple), making it inoperable. Other critical areas highlighted in different colors are Broca's Area (initiation of speech), in red and Wernike's Area (understanding of speech), in yellow.

FIG. 8, for a different patient, depicts a collection of three images. The image at upper left depicts a 3D reconstruction of a glioblastoma mass (GBM), in white, and a satellite tumor in yellow. This patient was previously treated for a GBM, shown encircled in white in the lower left image. However, at an unknown point in time, the patient had developed a small satellite tumor, encircled in yellow, right image, which was not treated. Highlighting of the cingulate tract, which serves memory and emotion functions, shows how the satellite tumor formed, i.e., cancerous cells travelled along the cingulate tract from the left frontal lobe GBM to the left temporal lobe to form the satellite tumor. These images graphically illustrate why it is not sufficient simply to draw a circle around a mass and blast it with radiation, which inherently is assuming that the mass (and all cancerous cells) have remained isotropic and that no cancerous cells have spread along any invisible pathways; rather, the design of the first treatment should have been anisotropic in nature, i.e., extended along the invisible pathways to kill any cancerous cells which may have spread and been in the process of forming satellite tumors.

FIG. 9 is a compound image, with the left 2D image depicting a horizontal slice through the chest and the right 3D image displaying a substantial portion of a patient's chest, which image has been focally altered to focus primarily upon what is important to the treatment planner. This patient has had a gastrectomy of the type known as a “gastrectomy, total” stomach removal, and re-attachment of the intestine via a method known as “Roux en Y,” which involves re-attaching two branches of the intestine (the “A-limb” and the “E-limb”) in a Y-shaped manner. In the image on the left, the color blue outlines the various structures shown in the sectional CT image. In the image on the right, the removed stomach is shown in yellow and in 3D, with red illustrating the “A” limb and blue illustrating the “E” limb. The program also automatically displays all these features not in just the 3D image but also in all of the 2D images of the CT scan which contain the features of interest.

There are five lobes of the lungs, with some 30-odd segments; separate cancers can develop in any or each of these segments. They obtain air through separate bronchial trees and have an autonomous vascular supply, but non-small-cell lung cancers often narrow or occlude a lobar airway (a bronchus), which often causes partial or complete collapse of the lung lobe or smaller segment within a lobe. If the patient is not strong enough to undergo a surgical resection of the affected lobe or segment, the cancer may be treated by radiation (radiotherapy), which typically has a number of important goals, such as minimizing damage to normal surrounding tissue and maximizing the radiation dose to the obstructing lung tumor. In addition, the secondarily collapsed but cancer-free lung or segment should not be irradiated, but involved nodes which contain gross cancer should be. (Gross cancer may be recognized by enlargement, by positive biopsy or by uptake on functional images.) Since the literature has shown that certain specific nodes often contain cancer cells, those specific nodes are preferably irradiated as well (“elective nodal irradiation”) because minimal nodal involvement can be missed with just traditional testing and traditional treatment (“involved nodal irradiation”).

Those skilled in the art will appreciate not only the enormity of the effort required to condense difficult medical literature (in this example, that of the bronchoscopy literature) and treatment details for any location and stage of lung cancer, but also the ease with such information may be understood and the rapidity with which it is presented. Not only does this advancement improve lung cancer treatment planning by making it more accurate and faster, but, more importantly, such good planning spares more normal tissue, thereby allowing greater dosages to be delivered to those areas which need it the most.

The lungs are the subject of the images of FIG. 10, which is another compound image, 2D horizontal sectional CT slice on the left and 3D image generated by the program and database of the present invention on the right. The airways are depicted in the color orange, in both the 3D image and in all 2D images; the blue region outlined in red (left, 2D image) highlights that part of the airway which supplies the right lung.

FIG. 11 is similarly a compound image, with 2D horizontal sectional CT slice on the left and the 3D image generated by the program and database on the right. Locations of the lymph nodes and respiratory tract are clearly shown.

FIG. 12 is similarly a compound image, with 2D horizontal sectional CT slice on the left and the 3D image generated by the invention on the right. The light blue highlights a normal, expanded right upper lung lobe, in both the 2D slices and in the dynamic, focally-altered 3D image; the dark blue correspondingly highlights a segment of a right upper lobe which has collapsed due to an airway lung cancer. The color yellow in all these images indicates areas for treatment avoidance: the large lower yellow structure, the heart; the longer elongated yellow structure, the spinal cord; the shorter elongated yellow structure, the esophagus; the horizontal yellow structures, the nerves transiting through the arm pit (brachial plexus). Airways in the vicinity are depicted in orange.

With these images—FIGS. 10 through 12—the planning physician can more accurately target the tumor and reduce the dose to normal tissue.

FIG. 13 depicts four images, two normal images on the left, and on the right, two images altered to depict the effects of the particular disease, pancreatic cancer. Careful study will show that the tumor (darker gray area) is confined to the head of the gland which obstructs the outflow of pancreatic fluid and bile, causing abnormal enlargement of the pancreatic and common bile ducts.

What is thus provided are novel means and methods for dramatically improving the standard of medical care throughout the nation. With widespread adoption and use of the present invention, medical professionals practicing far from the leading research centers will be able to deliver just as high a quality of care as can those few physicians having access to the latest but extremely expensive equipment, and at far less cost. For example, literally having at their fingertips the ability to view all functional tracts of the brain, immediately and on demand, in conjunction with images of tumors, will permit any physician, at any location, to accurately ascertain whether a given tumor is or is not operable or treatable, and if it is, to devise the best treatment plan possible. Similarly, the remote physician will no longer be handicapped by having limited access to reference libraries, and in fact will have access to greater reference sources than the typical large medical center physician not utilizing the present invention, and in a vastly more convenient form that will save an immense amount of time and, hence, ultimately deliver better medical care at greatly reduced cost.

What is claimed as new and desired to be secured by Letters Patent of the United States is:

1. An improved method for generating an image of a body structure to be treated, wherein the improvement comprises:
generating within said image an image of at least one tumor in at least one said body structure; and
generating on demand an image of at least one functional pathway of interest to at least one said tumor.

2. The improved method of claim 1, wherein the improvement further comprises the step of generating on demand all functional pathways of interest to at least one said tumor.

3. The improved method of claim 1, further including the step of devising an anisotropic plan for treating said tumor which includes at least a portion of at least one functional pathway external to said tumor.

4. A method for improving standards of medical decision-making, comprising the steps of:
selecting relevant information from a first domain of medical literature and integrating said first selected information with selected information from a second domain of medical literature;
compiling said integrated information into a systematic format for presentation immediately and on demand; and
converting any of said information into a plurality of sectional images and at least one three-dimensional image.

5. The method of claim 4, wherein said systematic format comprises selected visual textual information.

6. The method of claim 5, further comprising the step of providing supplemental information immediately upon demand.

7. The method of claim 4, wherein said systematic format comprises a video library of movement disorders.

8. The method of claim 7, further comprising the step of presenting a brain image highlighting a specific brain region which correlates with a given movement disorder, immediately upon demand.

9. The method of claim 4, wherein said systematic format comprises a library of speech disorders.

10. The method of claim 9, further comprising the step of presenting a brain image highlighting a specific brain region which correlates with a given speech disorder, immediately upon demand.

11. The method of claim 4, wherein a plurality of said sectional images are dynamic images which may be sized, translated or rotated at will.

12. The method of claim 4, wherein at least one three-dimensional image is a dynamic image which may be sized, translated and rotated at will.

13. The method of claim 4, wherein any of said selected information is information of abnormalities.

14. The method of claim 4, wherein at least one said domain is the domain of tumor biology.

15. The method of claim 4, wherein any of said images may be overlayed with patient images.

16. The method of claim 4, further comprising the step of continuing a structure of interest intermediate a plurality of sectional images and displaying said continued structure in a dynamic three-dimensional image.

17. The method of claim 16, wherein said three-dimensional display is focally altered, whereby a user's attention may be more readily focused upon said structure of interest.

18. A computer-searchable database of abnormal conditions, comprising:
means for presenting an image of a body structure to be treated;
means for generating within said image an image of at least one tumor within at least one said body structure; and
means for generating on demand an image of at least one functional pathway of interest to at least one said tumor.

19. The computer-searchable database of claim 18, further comprising means for generating on demand all functional pathways of interest to at least one said tumor.

20. The computer-searchable database of claim 18, further comprising means for displaying an anisotropic treatment plan for at least one said tumor.

21. A system for improving standards of medical decision-making, comprising:
means for storing selected relevant information from a first domain of medical literature and means for storing information from a second domain of medical literature;
means for integrating said selected relevant information from said first domain with said selected relevant information from said second domain;
means for compiling said integrated information into a systematic format for presentation immediately and on demand; and
means for converting any of said information into a plurality of sectional images and at least one three-dimensional image.

22. The system of claim 21, wherein said systematic format comprises selected visual textual information.

23. The system of claim 22, further comprising means for providing supplemental information immediately upon demand.

24. The system of claim 21, wherein said systematic format comprises a video library of movement disorders.

25. The system of claim 24, further comprising means for presenting a brain image highlighting a specific brain region which correlates with a given movement disorder, immediately upon demand.

26. The system of claim 21, wherein say systematic format comprises a library of speech disorders.

27. The system of claim 26, further comprising means for presenting a brain image highlighting a specific brain region which correlates with a given speech disorder, immediately upon demand.

28. The system of claim 21, wherein a plurality of said sectional images are dynamic images which may be sized, translated or rotated at will.

29. The system of claim 21, wherein any of said selected information is information of abnormalities.

30. The system of claim 21, wherein at least one said domain is the domain of tumor biology.

31. The system of claim 21, wherein any of said images may be overlayed with patient images.

32. The system of claim 21, further comprising means for continuing a structure of interest intermediate a plurality of sectional images and displaying said continued structure in a dynamic three-dimensional image.

33. The system of claim 32, wherein said three-dimensional display is focally altered, whereby a user's attention may be more readily focused upon said structure of interest.
